(19) United States
(12) Patent Application Publication
(10) Pub. No.: US 2014/0142175 A1
(43) Pub. Date: May 22, 2014

(54) ANHYDROUS COSMETIC COMPOSITIONS CONTAINING ASCORBIC ACID

(71) Applicant: L'OREAL S.A., Paris (FR)

(72) Inventors: Meriam Kelada, Bloomfield, NJ (US); Carol Elmasry, South Amboy, NJ (US)

(73) Assignee: L'Oreal S.A., Paris (FR)

(21) Appl. No.: 13/681,774

(22) Filed: Nov. 20, 2012

(51) Int. Cl. A61K 8/81; A61Q 19/08; A61Q 19/00; A61K 8/67

(52) U.S. Cl. CPC A61K 8/8182; A61K 8/676; A61Q 19/08; A61Q 19/00
USPC 514/474

(57) ABSTRACT

The disclosure relates to cosmetic compositions comprising ascorbic acid, at least two polyols, and a copolymer, wherein the composition is essentially anhydrous. The cosmetic compositions are particularly suitable for use on the face of a consumer, for example, as a facial mask. The disclosure also provides methods for improving the appearance of skin, for delivering ascorbic acid to the skin, and/or for reducing the effects and appearance of aging using the cosmetic compositions described herein.

### TECHNICAL FIELD

The present disclosure relates to compositions and methods for delivering ascorbic acid to the skin. Ascorbic acid is known to improve the appearance of facial skin and reduce the effects of aging.

### BACKGROUND

Consumers are interested in finding ways to minimize the aging process, especially its effects on personal appearance. To achieve the goal of looking healthy and youthful, consumers utilize skin care products, diet supplements, beautification methods and other means to slow down the aging process. In addition, consumers also seek to reduce the effects of sunlight on the skin that include dark spots, wrinkling, and other signs of aging.

Ascorbic acid is known for its positive impacts on the skin. As an antioxidant, ascorbic acid protects the skin against free radicals that come from pollution and UV radiation. Ascorbic acid is an enhancer of the biosynthesis of collagen, which is the protein that provides shape and firmness to the skin. Because ascorbic acid leads to higher production of collagen, wrinkling in skin is minimized. In addition, ascorbic acid is known to decrease age spots by reversing hyperpigmentation in skin cells to a certain extent by inhibiting tyrosinase activity.

The various effects of ascorbic acid to the aging process of the skin have been substantiated by researchers. As an example, a clinical study described by Humbert et al. was conducted to evaluate whether there was an improvement to photo-induced aging to the face by ascorbic acid (Exp. Dermatol. 2003, 12, 237-244). This double-blind, randomized trial was performed for a 6-month period over the effects of ascorbic acid versus those of an excipient. It was found that, upon daily application of ascorbic acid, significant improvements to the photo-induced aging skin were achieved, such as an increase in the density of the skin micro-reliefs and a decrease of the deep furrows.

Various approaches to deliver ascorbic acid as a skin care treatment have been reported. For instance, U.S. Pat. No. 5,736,567 describes an aqueous composition comprising ascorbic acid dissolved in water and a polyol. Another example is U.S. Pat. No. 6,087,393, which describes a topical use of ascorbic acid utilizing a mixed glycol carrier to stabilize ascorbic acid from oxidation.

Another method of providing ascorbic acid topically is a two component system in which the components must be mixed immediately before the consumer can apply the product to the skin. For instance, the product known as “Philosophy Turbo C Powder” requires the step of mixing the powder that contains ascorbic acid into a liquid. However, consumers desire a more convenient approach where the product can be applied directly to the skin without any premixing. Similarly, some products are “all-in-one” products that do not require premixing. However, these products are often aqueous and have an acidic pH, which is not well tolerated by those with sensitive skin.

### BRIEF SUMMARY OF THE DISCLOSURE

The present disclosure is directed to compositions and methods that are unique in comparison to conventional compositions and methods because they deliver a high concentration of ascorbic acid to the skin. Ascorbic acid is very difficult to stabilize and therefore can be challenging to formulate. Furthermore, the compositions and methods do not leave unwanted residue on the face even though they contain copolymers, often in high amounts. When copolymers are used at high concentrations, they have a tendency to leave a sticky residue on the body. Finally, the compositions are unique in that they are anhydrous. It is unusual to formulate and deliver ascorbic acid via an anhydrous carrier.

One aspect of the present disclosure is directed an anhydrous composition of ascorbic acid for a facial cosmetic product. The composition comprises ascorbic acid, at least one or two polyols, a copolymer, and is essentially anhydrous. The ascorbic acid may be present in an amount of from about 1 to 50% by weight, from about 10 to 30% by weight, or at about 15% by weight, based on the total weight of the composition.

The at least one or two polyols is/are typically selected from the group consisting of ethylene glycol, 1,2-propanediol, 1,3-propanediol, 1,2,3-propanetriol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

Alternatively, the at least two polyols include 1,2,3-propanetriol and at least one polyol selected from the group consisting of ethylene glycol, 1,2-propanediol, 1,3-propanediol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

In another embodiment, the at least two polyols include 1,2-propanediol and at least one polyol selected from the group consisting of ethylene glycol, 1,3-propanediol, 1,2,3-propanetriol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

In some embodiments, the at least two polyols include 1,2,3-propanetriol and 1,2-propanediol.

The one or more polyols are often in an amount of from about 35 to 98% by weight, from about 45 to 85% by weight, or at about 75% by weight, based on the total weight of the composition. In one embodiment, the at least two polyols include 1,2,3-propanetriol and 1,2-propanediol and are in a ratio of from about 1:1 to about 1:20, from about 1:5 to about 1:15, or about 1:10 (1,2,3-propanetriol:1,2-propanediol).

The copolymer is typically a film-forming copolymer, i.e., it forms an adhesive layer on skin. For instance, examples of film forming copolymers appropriate for use as described herein include at least one member selected from the group consisting of polyvinylpyrrolidone/vinyl acetate copolymer, hydrogenated styrene/isoprene copolymer, styrene/polyvinylpyrrolidone copolymer, acrylates copolymer, vinylpyrrolidone/hexadecane copolymer, vinylpyrrolidone/eicosene copolymer, vinylpyrrolidone/acrylates/lauryl methacrylate copolymer, vinyl caprolactam/vinylpyrrolidone/dimethylaminoethyl methacrylate copolymer, vinylpyrrolidone/dimethylaminoethyl methacrylate copolymer, acrylates/octylacrylamide copolymer, polisilicone-8(3-Thiopropyl methyl siloxane, polymer with dimethylsiloxane, S-ester with polymer of 2-propenoic acid and methyl 2-methyl-2-propenoate), vinylacetate/crotonates/vinyl neodecanoate copolymer, octylacrylamide/acrylates/butylaminoethyl methacrylate copolymer, and sodium polystyrene sulfonates.

In some embodiments, the copolymer is a polyvinylpyrrolidone/vinyl acetate copolymer.

Typically, the amount of copolymer ranges from about 1 to 20% by weight, from about 2 to 15% by weight, or is at about 10% by weight, based on the total weight of the composition.

In one embodiment, the amount of ascorbic acid ranges from about 1 to 50% by weight; the amount of the at least two polyols ranges from about 35 to 98% by weight; and the amount of the copolymer ranges from about 1 to 20% by weight, wherein the weight percentages are based on the total weight of the composition.

In one embodiment, the at least two polyols include 1,2,3-propanetriol and 1,2-propanediol, and the copolymer is polyvinylpyrrolidone/vinyl acetate copolymer. In another embodiment, ascorbic acid is in an amount of from about 1 to 50% by weight; 1,2,3-propanetriol and 1,2-propanediol is in an amount of from about 35 to 98% by weight; and polyvinylpyrrolidone/vinyl acetate copolymer is in an amount of from about 1 to 20% by weight, wherein the weight percentages are based on the total weight of the composition.

In some embodiments, the compositions comprise about 15% by weight of ascorbic acid, about 75% by weight of 1,2,3-propanetriol and 1,2-propanediol, and about 10% by weight of polyvinylpyrrolidone/vinyl acetate copolymer, wherein the weight percentages are based on the total weight of the composition.

The compositions described herein may be formulated as a cosmetic facial mask, comprising ascorbic acid; at least two polyols; and a copolymer, wherein the facial mask is essentially anhydrous. In some embodiments, the at least two polyols include 1,2,3-propanetriol and 1, 2-propanediol, and the copolymer is polyvinylpyrrolidone/vinyl acetate copolymer. Typically, the amount of ascorbic acid ranges from about 1 to 50% by weight, the amount of 1,2,3-propanetriol and 1,2-propanediol range from about 35 to 98% by weight, and the amount of polyvinylpyrrolidone/vinyl acetate copolymer ranges from about 1 to 20% by weight, wherein the weight percentages are based on the total weight of the composition.

In some embodiments, ascorbic acid is in an amount of about 15% by weight, the 1,2,3-propanetriol and 1,2-propanediol is in an amount of about 75% by weight, and the polyvinylpyrrolidone/vinyl acetate copolymer is in an amount of about 10% by weight, wherein the weight percentages are based on the total weight of the cosmetic mask.

Another aspect of the present disclosure is a method for using a composition or cosmetic facial mask as described herein to improve the appearance of facial skin, to deliver ascorbic acid to the facial skin, and/or to reduce the effects and appearance of aging. In one embodiment, the method comprises:
i. applying a compositions or facial mask as described herein to the face;
ii. allowing the composition or facial mask to remain on the face for finite amount of time; and
iii. removing the composition or facial mask from the face.

The facial mask it typically removed by washing the face or by rinsing the face with water after a finite amount of time. The finite amount of time may be about 5 minutes, about 10, minutes, about 15, minutes, about 20 minutes, about 25 minutes, about 30 minutes, or longer). In one embodiment, the finite amount of time is overnight. The facial mask may be applied to the face before going to sleep and is removed upon waking, typically the following morning.

The anhydrous ascorbic acid compositions of the present disclosure provide several desirable advantages from the perspective of the consumer. First, the compositions are stable. By utilizing a combination of the polyols and copolymer, the ascorbic acid remains in a stable form.

The compositions also advantageous for delivering ascorbic acid in an all-in-one product. Consumers can use it as a one-step skin care product, which is more convenient than other formulations that require an extra step of mixing ascorbic acid into a liquid carrier for application to the skin.

As mentioned above, the compositions of the present disclosure are essentially anhydrous. By avoiding the use of water, concerns with exposing the face to a low pH solutions is circumvented. In addition, the absence of water in the composition contributes to the stability of ascorbic acid. This allows for products comprising the compositions to enjoy a surprisingly long shelf-life.

### BRIEF DESCRIPTION OF THE DRAWINGS

FIG. 1 is a graphical representation of a stability test in which the slope of the line corresponds to the rate of degradation of the ascorbic acid.

FIG. 2 is a graphical representation of the results of the stability test in the form of a bar graph.

### DETAILED DESCRIPTION OF THE DISCLOSURE

As used herein, the term “a” and the phrase “at least one” refers to one or more and thus includes individual components as well as mixtures or combinations.

As used herein, the term “ascorbic acid” refers to ascorbic acid, L-ascorbic acid, vitamin C, or combinations thereof.

As used herein, the term “polyol” refers to a compound containing at least two hydroxyl groups.

As used herein, the term “anhydrous” refers a composition which is free of water. As used herein, the phrase “essentially anhydrous” refers to a composition which may contain a small amount of water such that the water does not materially affect the stability of ascorbic acid or otherwise materially affect the basic and novel characteristics of the present disclosure. For example, a composition which is “essentially anhydrous” may contain water in an amount of less than about 2.0% by weight, less than about 1.5% by weight, less than about 1.0% by weight, less than about 0.5% by weight, less than about 0.1% by weight, based on the total weight of the composition.

A first aspect of the present disclosure is directed to an essentially anhydrous cosmetic composition which contains ascorbic acid, at least one or two or more different polyols, and a copolymer.

The ascorbic acid may be present in an amount of from about 1 to about 50% by weight, from about 5 to about 45% by weight, from about 10 to about 30% by weight, from about 15 to about 25% by weight, or is in an amount of about 15% by weight, based on the total weight of the composition.

The one, two, or more polyols typically have a C₂-C₅ carbon chain. Examples of polyols include, but are not limited to, ethylene glycol, 1,2-propanediol, 1,3-propanediol, 1,2,3-propanetriol (“glycerin”), 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

In certain embodiments, the at least two polyols include 1,2,3-propanetriol and at least one polyol selected from the group consisting of ethylene glycol, 1,2-propanediol, 1,3-propanediol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

In another embodiment, the at least two polyols include 1,2-propanediol and at least one polyol selected from the group consisting of ethylene glycol, 1,3-propanediol, 1,2,3-propanetriol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

In a particular embodiment, the at least two different polyols include 1,2,3-propanetriol and 1,2-propanediol.

The at least one, two, or more polyols are typically present in the cosmetic composition in an amount of from about 35 to about 98% by weight, from about 40 to about 95% by weight, from about 45 to about 85% by weight, from about 50 to about 80% by weight, or may be present in an amount of 75% by weight, based on the total weight of the composition.

The one, two, or more polyols may be present in varying ratios in relation to each other. For instance, the ratio of a first polyol, for example, 1,2,3-propanetriol, to a second polyol, for example, 1,2-propanediol, may be from about 1:1 to about 1:20, from about 1:5 to about 1:15, or is about 1:10.

The copolymer is typically a film-forming polymer. The term “film-forming” means that the copolymer is capable of forming an adhesive layer on the skin. Additionally, the film forming polymer in some instances is capable of forming, alone or in the presence of an optional plasticizer, an isolatable film. Examples of film-forming copolymers useful herein include polyvinylpyrrolidone/vinyl acetate copolymer, hydrogenated styrene/isoprene copolymer, styrene/polyvinylpyrrolidone copolymer, acrylates copolymer, vinylpyrrolidone/hexadecane copolymer, vinylpyrrolidone/eicosene copolymer, vinylpyrrolidone/acrylates/lauryl methacrylate copolymer, vinyl caprolactam/vinylpyrrolidone/dimethylaminoethyl methacrylate copolymer, vinylpyrrolidone/dimethylaminoethyl methacrylate copolymer, acrylates/octylacrylamide copolymer, polisilicone-8, vinylacetate/crotonates/vinyl neodecanoate copolymer, octylacrylamide/acrylates/butylaminoethyl methacrylate copolymer, and sodium polystyrene sulfonates.

In some embodiments, the copolymer is polyvinylpyrrolidone/vinyl acetate, which is commonly known as a VP/VA copolymer. The VP/VA copolymer is not normally used for cosmetic compositions for the skin, particularly for facial composition, because it can be difficult to remove from the skin and sometimes leaves an unwanted residue. Moreover, the film that VP/VA copolymer often imparts to the skin is not typically comfortable to end users. However, the inventors discovered the VP/VA copolymer, when used with the at least two different polyols, stabilizes ascorbic acid in the present composition. Although the mechanism has not been elucidated yet, it is possible that the VP/VA copolymer and the at least two different polyols, for example 1,2,3-propanetriol and 1,2-propanediol, allow ascorbic acid to remain suspended, even after prolonged storage, thereby increasing stability (i.e., preventing precipitation). Moreover, it was also unexpectedly found that the compositions are easy to wash away from skin, and tolerable on the skin of consumers, even consumers with sensitive skin.

The copolymer may be present in an amount of from about 1 to about 20% by weight, from about 2 to about 18% by weight, from about 2 to about 16% by weight, from about 3 to about 15% by weight, from about 5 to about 12% by weight, or is present in an amount of about 10% by weight, based on the total weight of the composition.

In one embodiment, the ascorbic acid ranges from about 1 to 50% by weight, the at least two polyols range from about 35 to 98% by weight, and the copolymer ranges from about 1 to 20% by weight, wherein the weight percentages are based on the total weight of the composition. In another embodiment, ascorbic acid ranges from about 1 to 50% by weight, 1,2,3-propanetriol and 1,2-propanediol range from about 35 to 98% by weight, and the polyvinylpyrrolidone/vinyl acetate copolymer ranges from about 1 to 20% by weight, wherein the weight percentages are based on the total weight of the composition. In yet another embodiment, ascorbic acid is present at about 15% by weight, 1,2,3-propanetriol and 1,2-propanediol combined are at about 75% by weight, and polyvinylpyrrolidone/vinyl acetate copolymer is at about 10% by weight, wherein the weight percentages are based on the total weight of the composition.

The present disclosure is also directed to a cosmetic facial mask comprising a cosmetic composition described herein. The term “cosmetic facial mask” includes various forms of facial masks that can be applied to the face of consumers. A typical form of a facial mask can be a sheet-type made of either paper or cloth that is applied to the face. Another form of facial mask can be a peel, cream, or clay that is applied to the face of the consumer.

The present disclosure is also directed to a method for using a composition or cosmetic facial mask as described herein to improve the appearance of facial skin, to deliver ascorbic acid to the facial skin, and/or to reduce the effects and appearance of aging. In one embodiment, the method comprises:
i. applying a compositions or facial mask as described herein to the face;
ii. allowing the composition or facial mask to remain on the face for finite amount of time; and
iii. removing the composition or facial mask from the face.

The facial mask it typically removed by washing the face or by rinsing the face with water after a finite amount of time. The finite amount of time may be about 5 minutes, about 10, minutes, about 15, minutes, about 20 minutes, about 25 minutes, about 30 minutes, or longer). In one embodiment, the finite amount of time is overnight. The facial mask may be applied to the face before going to sleep and is removed upon waking, typically the following morning.

The compositions described herein are typically in the form of a ready-to-use product and are essentially anhydrous. The term “ready-to-use” means that the product is to be directly applied to a user's skin, particularly a user's face, without requiring premixing of any of the components of the composition before application to the skin.

The compositions can optionally comprise additional components such as skin tone changing agents, whitening agents, anti-inflammatory agents, antimicrobial agents, skin texture improvement agents, and additional skin benefit agents. These additional components are typically in an amount of about 0.001, 0.01, 0.1, 1.0, or 2.0% to about 3, 5, 10, 15, 20, 30, 40, or 50% by weight, based on the total weight of the composition.

### Skin Tone Changing Agent

The compositions of the present disclosure may include a skin tone changing agent such as skin tone changing pigments, reflective particulate material, and mixtures thereof. Skin tone changing agents useful herein are those altering the appearance of the color and/or tone of the skin including, but not limited to, skin whitening. The skin tone changing agents can have a particle size of at least about 100 nm. Examples of skin tone changing pigments useful herein include, for example, talc, mica, silica, magnesium silicate, titanium oxide, zinc oxide, and titanium oxide coated mica.

The reflective particulate materials useful herein typically have a primary particle size of from about 100 nm to about 10 um (i.e., in the essentially pure, powder form prior to combination with any carrier). The reflective particulate materials can be inorganic. The inorganic reflective particulate materials include, for example, titanium dioxide, zinc oxide, and particles that consist essentially of titanium dioxide. The inorganic reflective particulate materials can be coated with a coating material such as cationic polymers, cationic surfactants, anionic polymers, and anionic surfactants.

### Chronic Whitening Agent

The compositions may include a chronic whitening agent. A chronic whitening agent is an active ingredient that not only alters the appearance of the skin, but further improves hyperpigmentation as compared to pre-treatment. By definition, chronic is referred to continued topical application of the composition over an extended period, e.g., for a period of at least about one week, for a period of at least about one month, for at least about three months, or for at least about one year. Typically, applications would be on the order of about once per day over such extended periods, while application rates can vary from about once per week, twice per week, three times per week, twice per day, or three times per day or more. The chronic whitening agents may be included, by weight of the composition, at a level from about 0.001% to about 10%, or from about 0.1% to about 5%.

Useful chronic whitening agents include vitamin B3 compounds, azelaic acid, butyl hydroxy anisole, gallic acid and its derivatives, glycyrrhizinic acid, hydroquinoine, kojic acid, arbutin, mulberry extract, ergothioneine, and mixtures thereof.

### Anti-Inflammatory Agents

Anti-inflammatory agents enhance the skin appearance benefits, by for example, contribution of uniformity and acceptable skin tone and/or color. Typically, the anti-inflammatory agent includes a steroidal anti-inflammatory agent and a non-steroidal anti-inflammatory agent. In one embodiment, the steroidal anti-inflammatory for use is hydrocortisone.

So-called “natural” anti-inflammatory agents are also useful. Such agents may suitably be obtained as an extract by suitable physical and/or chemical isolation from natural sources (i.e., plants, fungi, by-products of microorganisms). For example, alpha bisabolol, aloe vera, Manjistha (extracted from plants in the genus Rubia, particularly Rubia Cordifolia), and Guggal. (extracred from plants in the genus Commiphora, particularly Commiphora Mukul), kola extract, chamomile, and sea whip extract, may be used.

Additional anti-inflammatory agents useful herein include compounds of the licorice (the plant genus/species Glycyrrhiza glabra) family, including glycyrrhetic acid, glycyrrhizic acid, and derivatives thereof (e.g. salts and esters). Suitable salts of the foregoing compounds include metal and ammonium salts, Suitable esters include C₂₋₂₄ saturated or unsaturated esters of the acids, C₁₀₋₂₄, or C₁₆₋₂₄.

### Antimicrobial Agents

As used, “antimicrobial agents” means a compound capable of destroying microbes preventing the development of microbes or preventing the pathogenic action of microbes. Antimicrobial agents are useful, for example, in controlling acne. Some antimicrobial agents useful in the present disclosure are benzoyl peroxide, erythromycin, tetracycline, clindamycin, azelaic acid, sulfur resorcinol phenoxyethanol, and IRGASAN™ DP300 (Ciba Geigy Corp., U.S.A). A safe and effective amount of an antimicrobial agent may be added to compositions of the present disclosure, typically from about 0.001% to about 10%, from about 0.01% to about 5%, or from about 0.05% to about 2%.

### Skin Texture Improvement Agents

Skin texture treatment agents help repair and replenish the natural moisture barer function of the epidermis, thereby providing skin benefits such as texture improvement.

Skin texture improvement agents useful herein are niacinamide, nicotinic acid and its esters, nicotinyl alcohol, panthenol, panthenyl ethyl ether, n-acetyl cysteine, n-acetyl-L-serine, phosphodiesterase inhibitors, trimethyl glycine, tocopheryl nicotinate, and vitamin B.sub.3 and analogues or derivatives, and mixtures thereof. Panthenol is particularly preferred in that, when used in an amount of at least about 1%, it provides texture improvement benefits. Panthenol is commercially available, for example, by Roche.

### Other Components

In addition to the above described components, the composition of the present disclosure may further include ultraviolet light absorbers or scattering agents; sequestrants; anti-androgens; depilation agents; soluble or colloidally-soluble moisturizing agents such as hyaluronic acid and starch-grafted sodium polyacrylates such as SANWET™ IM-1000, IM-1500 and IM-2500 available from Celanese Superabsorbent Materials, Portsmith, Va., USA and described in U.S. Pat. No. 4,076,663; proteins and polypeptides and derivatives thereof; organic hydroxy acids; drug astringents; external analgesics; film formers; anticaking agents; antifoaming agents; binders; coloring agents; perfumes, essential oils, and solubilizers thereof; natural extracts; guai-azulene; and yeast ferment filtrate.

Aspects of the present disclosure are illustrated in greater details in the following examples, which are exemplary and not limiting.

### EXAMPLE 1

### Preparation of Formulations

Formulations 1 and 2 of the present disclosure were prepared by mixing the components identified according to Table 1 below.

TABLE 1

| | | Comparative |
|---|---|---|
| Component | Formulation 1 | Formulation 1 |
| Ascorbic Acid | 15% | 15% |
| 1,2,3-Propanetriol and | 74.95% | 85% |
| 1,2-Propanediol | | |
| Polyvinylpyrrolidone/Vinyl | 10% | 0% |
| Acetate Copolymer | | |
| Additive | 0.04% | 0.04% |
| (Adenosine (anti-wrinkle | | |
| compound)) | | |

Comparative Formulation 1 was prepared without the polyvinylpyrrolidone/vinyl acetate copolymer to compare and analyze the effects of the copolymer on the product.

### EXAMPLE 2

### Home Flash Consumer Test of Formulation 1

To obtain consumers' opinion, perceptions and overall satisfaction with Formulation 1, 18 women subjects participated in a home flash evaluation in which the subjects responded to semi-qualitative questionnaire with closed questions.

All of the subjects were over the age of 30 with self-perceived “normal to dry” facial skin. Each subject also used a skincare product (i.e., facial moisturizer, serum, and/or mask) containing ascorbic acid (Vitamin C) at least two times per week. The test product was presented in a generic, blinded squeeze-tube bottle. The subjects were asked to self-apply the test at home for 14 days with no more than 2 applications per week. The instructions for application of the test product were to apply a thin layer of the test product all over the face, leave the test product on for 5 minutes, then rinse off with water. The subjects were also instructed to avoid contact with the eyes and to rinse off immediately if contact with the eyes occurred. The parameters with regard to texture, application, efficacy, fragrance and overall satisfaction were evaluated and the results are provided below in Table 2.

TABLE 2

| Portions of | |
|---|---|
| Subjects | Formulation 1 |
| All to Most | Appropriate color for a facial mask (i.e., clear), |
| Subjects | pleasant texture, smooth texture, greasy/non-greasy |
| | texture, easy application, absorption rate, easy rinsing, |
| | light skin feel, no residue, pleasantness of scent (i.e., |
| | non-descript, light), greasy/non-greasy skin feel, |
| | sticky/non-sticky skin feel, smooth skin feel, adequate |
| | moisturization, shiny to matte skin appearance, |
| | comfortable skin feel, overall satisfaction, and no |
| | uncomfortable sensations. |
| Few Subjects | Thin consistency |

Formulation 1 of the present disclosure received high overall satisfaction amongst the subjects, where 14 of the 18 subjects indicated that they were somewhat/very satisfied with the test product. Moreover, 16 of the 18 subjects indicated that the test product produced no uncomfortable sensations and/or adverse events. Two panelists noted minor sensations with the test product. Overall performance of the test product was favorable for nearly all visual and tactile attributes, including: appropriate color for a facial mask, texture (smooth, greasy/non-greasy), easy application, absorption rate, easy rinsing, tactile skin feel (light, greasy/non-greasy, sticky/non-sticky, smooth, comfortable), adequate moisturization, and scent (non-descript, light). However, few subjects were dissatisfied with the test product for its thin consistency, which they correlated to a “messy” application.

### EXAMPLE 3

### Comparison of Formulation 1 and Comparative Formulation 1

To obtain consumers' opinion, perception, satisfaction and overall preference between Formulation 1 and Comparative Formulation 1,12 women subjects participated in a home flash evaluation in which the subjects responded to a semi-qualitative questionnaire with closed questions.

All the subjects perceived themselves to have normal to dry facial skin, and they regularly used a luxury brand cosmetic facial masks 1-2 times per week. The test products were presented to the subjects in blinded, white, generic squeeze tubes. The subjects were asked to self-apply on facial skin at home one time during a 3-day period. The results of the comparison are presented in Table 3 below, which provides the portion of subjects that indicated the specific parameters concerning the formulations tested.

TABLE 3

| Portions of | | |
|---|---|---|
| Subjects | Formulation 1 | Comparative Formulation 1 |
| All to Most | Pleasantness of scent (i.e., no scent), | Pleasantness of scent (i.e., no scent), |
| Subjects | pleasant texture, smooth texture, | smooth texture, easy rinsing, no |
| | easy application, sensations of heat | residue, non-greasy skin feel, non- |
| | and tingling, comfortable skin feel | sticky skin feel, smooth skin feel, tight |
| | during application, easy rinsing, no | to not tight skin feel, clean skin feel, |
| | residue, non-greasy skin feel, non- | uncomfortable skin feel during |
| | sticky skin feel, smooth skin feel, | application (i.e., tingling and |
| | clean skin feel, comfortable skin feel | stinging), and radiant skin appearance. |
| | after application, no irritation, and | |
| | satiny/shiny/matte skin appearance. | |
| More Than | No improvement in appearance of | Too thin consistency |
| Half of the | fine lines and wrinkles. | |
| Subjects | | |
| Half of the | Too thick consistency | Unpleasant texture, inadequate |
| Subjects | | moisturization, no improvement in |
| | | appearance of fine lines and wrinkles |
| Few | Inappropriate color, not tight skin feel, | Inappropriate color, greasy texture, |
| Subjects | inadequate moisturization, not | no improvement in overall skin |
| | improved overall skin appearance | appearance, overall satisfaction |

Formulation 1 of the present disclosure received average satisfaction ratings from the subjects, among which 8 out of 12 subjects were somewhat satisfied to very satisfied. In contrast, Comparative Formulation 1 received a below average satisfaction ratings from the subjects, among which, 6 out of 12 subjects were somewhat satisfied to very satisfied.

Among the appreciated attributes, differences were noted between formulations for certain visual and tactile attributes. Formulation 1 was found to have a more pleasant texture, easy application, heat and tingling sensations, comfortable skin feel during application, comfortable skin feel after application, and satiny/shiny/matte skin appearance. Comparative Formulation 1 was found to provide a tight skin feel to not tight skin feel and a radiant skin appearance. In particular, Formulation 1 has an easier application and is more comfortable on the skin than Comparative Formulation 1.

### EXAMPLE 4

### Rate of Degradation of Ascorbic Acid

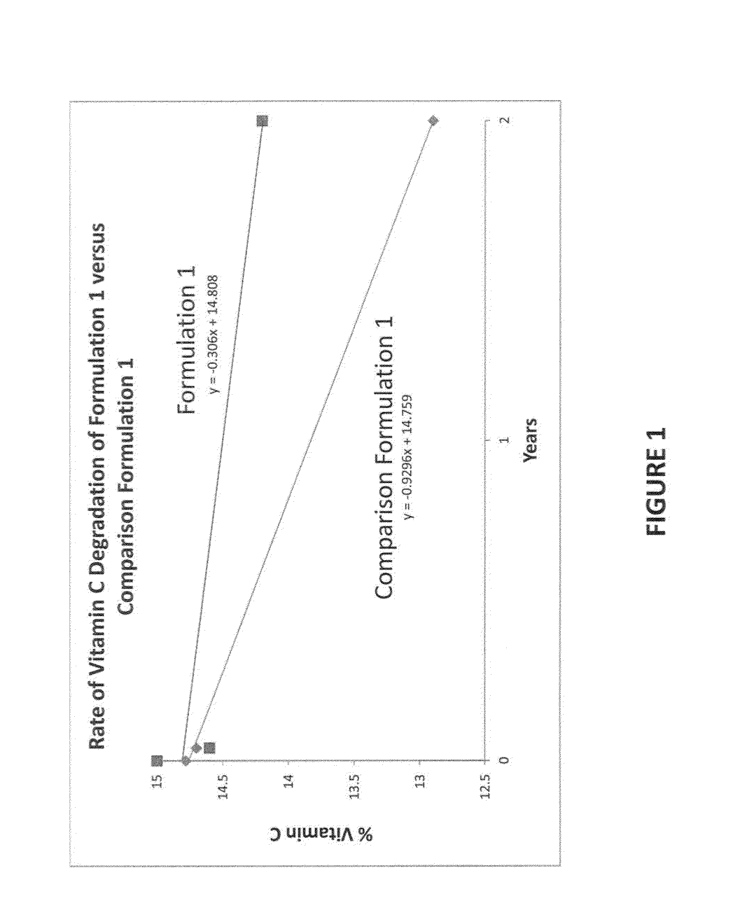

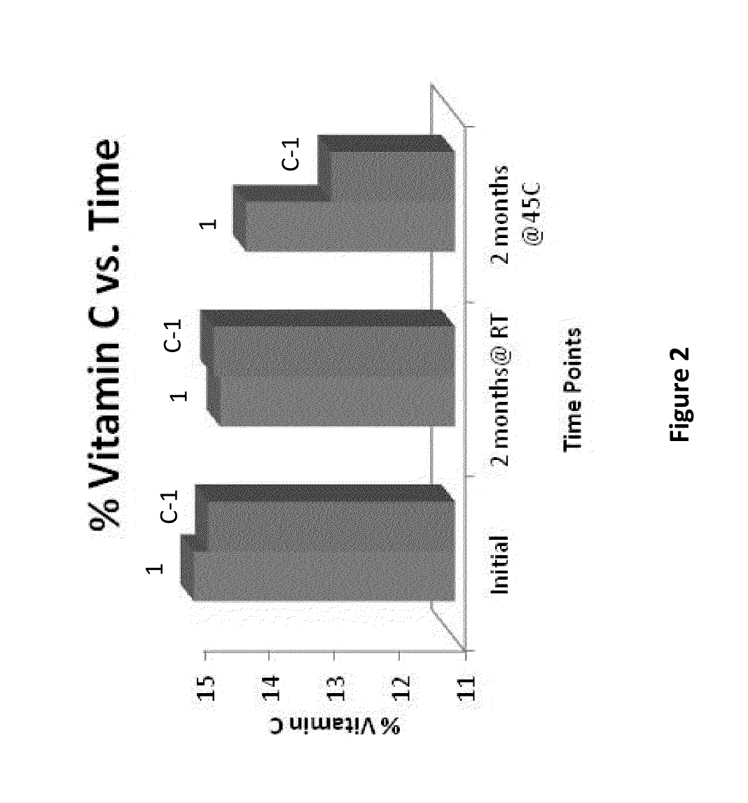

Formulation 1 and Comparison Formulation 1 were prepared as indicated above and the stability of the ascorbic acid (vitamin C) in the formulations was tested. Specifically, the amount of the ascorbic acid in the formulations was tested at an initial value, 2 months storage at room temperature and at 2 months storage at 45° C. FIG. 1 illustrates the results of the stability test in the form of a line graph in which the slope of the line corresponds to the rate of degradation of the ascorbic acid. As illustrated, Comparison Formulation 1 degraded three times faster than Formulation 1. FIG. 2 illustrates the results of the stability test in the form of a bar graph, which shows the percent of vitamin C degradation over time for Comparison Formulation 1 (C-1) and Formulation 1. The graph demonstrates that Formulation 1 degrades slower than Comparison Formulation 1 at 45° C. when stored for a period two month. Further, the graph demonstrates that the Formulation 1 and Comparison Formulation 1 degrade at approximately the same rate at room temperature when stored for a period of two months.

### EXAMPLE 5

### Visual Comparison of the Stability of Formulation 1 and Comparison Formulation 1

After completion of the storage times as indicated in Example 4, Formulation 1 and Comparison Formulation 1 were visually compared to determine the visual stability of the formulations. The results of the visual inspection are provided in Table 4 below.

TABLE 4

| | Visual Stability | |
|---|---|---|
| | 2 months storage at 37° C. | |
| Formulation | (room temperature) | 2 months storage at 45° C. |
| Formulation | Formulation appeared | Formulation appeared |
| 1 | clear with no evidence | clear with no evidence |
| | of precipitate or | of precipitate or |
| | crystallization. | crystallization. |
| | Slight yellowing observed. | Slight yellowing observed. |
| Comparison | Formulation appeared | Formulation appeared |
| Formulation | clear, but precipitate | clear, but precipitate |
| 1 | was observed. | was observed. |
| | Yellowing observed. | Yellowing observed. |

### EXAMPLE 6

### Consumer Study

To obtain consumers' opinion, perceptions and overall satisfaction with Formulation 1, 21 women subjects participated in a consumer study. All of the subjects were Caucasian and between the ages of 55 and 72. The subjects started by putting on a headband and washing their face in a conventional manner with Cetaphil® and water. Then, the subjects blotted their faces dry with laboratory wipes and waited for approximately 5 to 10 minutes. An image was then captured and followed by Cutometer, TEWL (transepidermal water loss) and hydration measurements. Cutometer readings were taken below the cheek bone, lateral to the nasolabial fold and measured elasticity. TEWL readings were taken from the cheek bone area below the temple to avoid air flow from the nose and mouth. Corneometer readings were taken from the cheek bone area below the eye and measured skin hydration. The subject's face was wiped with a small laboratory wipe prior to Corneometer readings as the Corneometer would only read the water content of the product and not the hydration of the skin if it is not wiped. The product was dosed onto a glass plate using a syringe and 0.3 mL was applied to each half of the face by a trained Cosmetologist, who ensures that application is even and well massaged into the skin. The subjects then rested for 15 minutes, and images were captured and readings were repeated.

When tested with a Cutometer®, Formulation 1 showed evidence of instant firming and elasticity improvements. Formulation 1 also increased skin hydration by 20% on the treated facial skin. TEWL, which is the measurement of the health of the skin barrier and increases when the skin is damaged or unhealthy, did not change and therefore Formulation 1 is safe or mild to the skin. Decreases in wrinkle volume were also observed. There was a 27% decrease in wrinkle volume, a 30% decrease in wrinkle area and a 28% decrease in the percentage of skin containing wrinkles. It is believed that the observed decrease in wrinkle attributes is due to a “plumping” of the skin related to hydration effects because it is not common to achieve similar results after leaving a product on the skin for only 5 minutes.

### EXAMPLE 7

### Consumer Test

To obtain consumers' opinions, perceptions, overall satisfaction and preference of Formulation 1 over time, 57 women subjects participated in a blinded at-home test for 4 weeks. All of the subjects were over the age of 35 with normal, normal-to-dry, or dry skin types. Each subject also used a luxury facial mask at least twice per month and had one or more of the following skin concerns: fine lines/wrinkles, age spots, and/or uneven skin tone.

The subjects were instructed to cleanse their face as their normally would, and then apply Formulation 1 to their entire face, while avoiding contact with the eyes. The subjects left the formulation on their face for 5 minutes and then rinsed their face with water. Finally, the subjects were instructed to apply the formulation twice per week.

The subjects provided responses to questions about Formulation 1. A Wilcoxon Signed Rank test was used to compare the attributes at the baseline (i.e., initial response) and week 2, as well as the baseline and week 4. When data passed normality testing, a paired test was used. The statistical significance level was set at a p-value of s 0.05. Significant differences were found for the majority of the attributes at both Week 2 and Week 4 when compared to the baseline.

As compared to the baseline after 2 weeks, the subjects felt that their skin felt smoother; firmer; softer; refreshed; more resilient/elastic; more moisturized; more comfortable; less tight; suppler; fine lines and wrinkles were less visible; skin appears more radiant, healthier, rested and younger. However, the subjects also felt that their skin was greasier and sticker/tackier, and that their complexion appeared less clear. Overall, the subjects felt that their skin quality appeared improved and the overall appearance of the skin improved.

As compared to the baseline after 4 weeks, the subjects felt that their skin felt smoother; firmer; suppler; more refreshed, resilient/elastic, moisturized and comfortable; less tight; fine lines and wrinkles appeared less visible; and skin appeared more radiant, healthier, more rested and younger. However, the subjects also felt that their skin was stickier and their complexion was less clear. Overall, the subjects felt that their skin quality appeared improved and the overall appearance of the skin improved. Thus, after both 2 and 4 weeks of using Formulation 1, the 57 subjects found significant improvements.

The consumers generally gave positive reactions to the use of Formulation 1. From the comparison between Formulation 1 and the Comparative Formulation 1, it can be observed that the use of a copolymer in Formulation 1 is helpful in minimizing much of the discomfort that consumers encountered using Comparative Formulation 1.

### Embodiments

Embodiment 1 is directed to a cosmetic composition comprising: ascorbic acid, one or more polyols, and a copolymer, wherein the composition is essentially anhydrous.

Embodiment 2 is directed to the cosmetic composition according to embodiment 1, wherein the ascorbic acid is present in an amount of from about 1 to about 50% by weight, based on the total weight of the composition.

Embodiment 3 is directed to the cosmetic composition according to Embodiments 1 or 2, wherein one or more polyols is selected from the group consisting of ethylene glycol, 1,2-propanediol, 1,3-propanediol, 1,2,3-propanetriol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

Embodiment 4 is directed to the cosmetic composition according to any one of embodiments 1-3, wherein the one or more polyols is selected from the group consisting of 1,2,3-propanetriol and a polyol selected from the group consisting of ethylene glycol, 1,2-propanediol, 1,3-propanediol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

Embodiment 5 is directed to the cosmetic composition according to any one of emobodiments 1-4, wherein composition comprises at least two different polyols, one polyol being 1,2-propanediol, and the other polylol selected from the group consisting of ethylene glycol, 1,3-propanediol, 1,2,3-propanetriol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

Embodiment 6 is directed to the cosmetic composition according to any one of embodiments 1-5, wherein the compositions comprises at least two polyols, and the at least two polyols include 1,2,3-propanetriol and 1,2-propanediol.

Embodiment 7 is directed to the cosmetic composition according to any one of embodiments 1-6, wherein the weight ratio of 1,2,3-propanetriol to 1, 2-propanediol is from about 1:1 to about 1:20.

Embodiment 8 is directed to the cosmetic composition according to any one of embodiments 1-7, wherein the weight ratio of 1,2,3-propanetriol to 1, 2-propanediol is about 1:10.

Embodiment 9 is directed to the cosmetic composition according to any one of embodiments 1-8, wherein the one or more polyols are present in an amount of from about 35 to about 98% by weight, based on the total weight of the composition.

Embodiment 10 is directed to the cosmetic composition according to any one of embodiments 1-9, wherein the copolymer is selected from the group consisting of polyvinylpyrrolidone/vinyl acetate copolymer, hydrogenated styrene/isoprene copolymer, styrene/polyvinylpyrrolidone copolymer, acrylates copolymer, vinylpyrrolidone/hexadecane copolymer, vinylpyrrolidone/eicosene copolymer, vinylpyrrolidone/acrylates/lauryl methacrylate copolymer, vinyl caprolactam/vinylpyrrolidone/dimethylaminoethyl methacrylate copolymer, vinylpyrrolidone/dimethylaminoethyl methacrylate copolymer, acrylates/octylacrylamide copolymer, polisilicone-8, vinylacetate/crotonates/vinyl neodecanoate copolymer, octylacrylamide/acrylates/butylaminoethyl methacrylate copolymer, sodium polystyrene sulfonates, and combinations thereof.

Embodiment 11 is directed to the cosmetic composition according to any one of embodiments 1-10, wherein the copolymer is polyvinylpyrrolidone/vinyl acetate copolymer.

Embodiment 12 is directed to the cosmetic composition according to any one of embodiments 1-11, wherein the copolymer is present in an amount of from about 1 to about 20% by weight, based on the total weight of the composition.

Embodiment 13 is directed to the cosmetic composition according to any one of embodiments 1-12, wherein the composition comprises: from about 1 to about 50% by weight of the ascorbic acid; from about 35 to about 98% by weight of one or more polyols; and from about 1 to about 20% by weight of the copolymer; wherein the weight percentages are based on the total weight of the composition.

Embodiment 14 is directed to the cosmetic composition according to any one of embodiments 1-14, comprising two polylols, wherein the polyols are 1,2,3-propanetriol and 1,2-propanediol; and wherein the copolymer is polyvinylpyrrolidone/vinyl acetate copolymer.

Embodiment 15 is directed to the cosmetic composition according to any one of embodiments 1-14, wherein the composition comprises: from about 1 to about 50% by weight of the ascorbic acid; from about 35 to about 98% by weight of the 1,2,3-propanetriol and 1,2-propanediol; and from about 1 to about 20% by weight of the polyvinylpyrrolidone/vinyl acetate copolymer; wherein the weight percentages are based on the total weight of the composition.

Embodiment 16 is directed to a cosmetic facial mask comprising the cosmetic composition according to any one of embodiments 1-15.

Embodiment 17 is directed to the cosmetic facial mask according to embodiment 16, comprising two polylols, wherein the polyols are 1,2,3-propanetriol and 1,2-propanediol; and wherein the copolymer is polyvinylpyrrolidone/vinyl acetate copolymer.

Embodiment 18 is directed to the cosmetic facial mask according to embodiment 17, wherein facial mask comprises: from about 1 to about 50% by weight of the ascorbic acid; from about 35 to about 98% by weight of the 1,2,3-propanetriol and 1,2-propanediol; and from about 1 to about 15% by weight of the polyvinylpyrrolidone/vinyl acetate copolymer; wherein the weight percentages are based on the total weight of the facial mask.

Embodiment 19 is directed to a method for improving the appearance of facial skin, for delivering ascorbic acid to the facial skin, and/or for reducing the effects and appearance of aging comprising: applying a cosmetic composition or mask according any one of embodiments 1-18 to the face; allowing the composition or mask to remain on the face for finite amount of time; and removing the composition or mask from the face.

Embodiment 20 is directed to a ready-to-use facial mask comprising a composition according to any one of embodiments 1-15.

What is claimed is:

1. A cosmetic composition comprising: ascorbic acid, one or more polyols, and a copolymer, wherein the composition is essentially anhydrous.

2. The cosmetic composition according to claim 1, wherein the ascorbic acid is present in an amount of from about 1 to about 50% by weight, based on the total weight of the composition.

3. The cosmetic composition according to claim 1, one or more polyols is selected from the group consisting of ethylene glycol, 1,2-propanediol, 1,3-propanediol, 1,2,3-propanetriol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

4. The cosmetic composition according to claim 1, wherein the one or more polyols is selected from the group consisting of 1,2,3-propanetriol and a polyol selected from the group consisting of ethylene glycol, 1,2-propanediol, 1,3-propanediol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

5. The cosmetic composition according to claim 1, wherein composition comprises at least two different polyols, one polyol being 1,2-propanediol, and the other polylol selected from the group consisting of ethylene glycol, 1,3-propanediol, 1,2,3-propanetriol, 1,2-butanediol, 1,3-butanediol, 1,4-butanediol, 1,2,3-butanetriol, 1,2,4-butanetriol, 1,2,3,4-butanetetrol, 1,2-pentanediol, 1,3-pentanediol, 1,4-pentanediol, 1,5-pentanediol, 2,3-pentanediol, 2,4-pentanediol, 1,2,3-pentanetriol, 1,2,4-pentanetriol, 1,2,5-pentanetriol, 2,3,4-pentanetriol, 1,2,3,4-pentanetetrol, 1,2,3,5-pentanetetrol and 1,2,3,4,5-pentanepentol.

6. The cosmetic composition according to claim 1, wherein the compositions comprises at least two polyols, and the at least two polyols include 1,2,3-propanetriol and 1,2-propanediol.

7. The cosmetic composition according to claim 6, wherein the weight ratio of 1,2,3-propanetriol to 1, 2-propanediol is from about 1:1 to about 1:20.

8. The cosmetic composition according to claim 6, wherein the weight ratio of 1,2,3-propanetriol to 1, 2-propanediol is about 1:10.

9. The cosmetic composition according to claim 1, wherein the one or more polyols are present in an amount of from about 35 to about 98% by weight, based on the total weight of the composition.

10. The cosmetic composition according to claim 1, wherein the copolymer is selected from the group consisting of polyvinylpyrrolidone/vinyl acetate copolymer, hydrogenated styrene/isoprene copolymer, styrene/polyvinylpyrrolidone copolymer, acrylates copolymer, vinylpyrrolidone/hexadecane copolymer, vinylpyrrolidone/eicosene copolymer, vinylpyrrolidone/acrylates/lauryl methacrylate copolymer, vinyl caprolactam/vinylpyrrolidone/dimethylaminoethyl methacrylate copolymer, vinylpyrrolidone/dimethylaminoethyl methacrylate copolymer, acrylates/octylacrylamide copolymer, polisilicone-8, vinylacetate/crotonates/vinyl neodecanoate copolymer, octylacrylamide/acrylates/butylaminoethyl methacrylate copolymer, sodium polystyrene sulfonates, and combinations thereof.

11. The cosmetic composition according to claim 1, wherein the copolymer is polyvinylpyrrolidone/vinyl acetate copolymer.

12. The cosmetic composition according to claim 1, wherein the copolymer is present in an amount of from about 1 to about 20% by weight, based on the total weight of the composition.

13. The cosmetic composition according to claim 1, wherein the composition comprises:
from about 1 to about 50% by weight of the ascorbic acid;
from about 35 to about 98% by weight of one or more polyols; and
from about 1 to about 20% by weight of the copolymer;
wherein the weight percentages are based on the total weight of the composition.

14. The cosmetic composition according to claim 1 comprising two polylols, wherein the polyols are 1,2,3-propanetriol and 1,2-propanediol; and wherein the copolymer is polyvinylpyrrolidone/vinyl acetate copolymer.

15. The cosmetic composition according to claim 14, wherein the composition comprises:
from about 1 to about 50% by weight of the ascorbic acid;
from about 35 to about 98% by weight of the 1,2,3-propanetriol and 1,2-propanediol; and
from about 1 to about 20% by weight of the polyvinylpyrrolidone/vinyl acetate copolymer;
wherein the weight percentages are based on the total weight of the composition.

16. A cosmetic facial mask comprising the cosmetic composition according to claim 1.

17. The cosmetic facial mask according to claim 16, womprising two polylols, wherein the polyols are 1,2,3-propanetriol and 1,2-propanediol; and wherein the copolymer is polyvinylpyrrolidone/vinyl acetate copolymer.

18. The cosmetic facial mask according to claim 17, wherein facial mask comprises:
from about 1 to about 50% by weight of the ascorbic acid;
from about 35 to about 98% by weight of the 1,2,3-propanetriol and 1,2-propanediol; and
from about 1 to about 15% by weight of the polyvinylpyrrolidone/vinyl acetate copolymer;
wherein the weight percentages are based on the total weight of the facial mask.

19. A method for improving the appearance of facial skin, for delivering ascorbic acid to the facial skin, and/or for reducing the effects and appearance of aging comprising:
i. applying a cosmetic composition according to claim 1 o the face;
ii. allowing the composition to remain on the face for finite amount of time; and
iii. removing the composition or facial mask from the face.

20. A ready-to-use facial mask comprising a composition according to claim 1.
